(19) United States
(12) Patent Application Publication
(10) Pub. No.: US 2021/0112995 A1
(43) Pub. Date: Apr. 22, 2021

(54) INTRAUTERINE ENVIRONMENT SIMULATION SYSTEM

(71) Applicant: ONTOGENETICS, INC., Penn Valley, CA (US)

(72) Inventor: Edward BUCKLEY, Penn Valley, CA (US)

(21) Appl. No.: 17/251,709

(22) PCT Filed: Jul. 9, 2019
(86) PCT No.: PCT/US2019/040932

§ 371 Date: Dec. 11, 2020

Related U.S. Application Data
(60) Provisional application No. 62/695,770, filed on Jul. 9, 2018.

(51) Int. Cl. A47D 9/02; A61M 21/00

(52) U.S. Cl. CPC A47D 9/02; A61M 21/00; A61M 2021/0027; A61M 2021/0022; A61M 2021/0061

(57) ABSTRACT

An intrauterine simulation system may comprise a motion-control system and/or a maternal heartbeat simulator. The motion-control system may be configured to move a platform supporting an infant in a pattern characteristic of the movement of a woman in a late stage of pregnancy, thereby simulating movement experienced by a fetus in the intrauterine environment. The maternal heartbeat simulator may be configured to produce low-frequency sound waves and/or vibrations and to transmit the sound waves and/or vibrations via the platform to the infant's body. This simulates the vibrations created by the mother's heart as experienced by a fetus. The platform supporting the infant may be incorporated into a bassinet, cradle, mattress, and/or other suitable device. A system controller may be configured to gradually reduce aspects of the simulation, such as the intensity of the sound waves and/or vibrations or extent of the movement, thereby transitioning an infant to the extrauterine environment.

### CROSS-REFERENCES

This application claims the benefit under 35 U.S.C. § 119(e) of the priority of U.S. Provisional Patent Application Ser. No. 62/695,770, filed Jul. 9, 2018, the entirety of which is hereby incorporated by reference for all purposes.

### FIELD

This disclosure relates to systems and methods for infant care. More specifically, the disclosed embodiments relate to infant bassinets, cradles, mattresses, pods, etc. configured to at least partially simulate aspects of an intrauterine environment.

### INTRODUCTION

Perhaps the most difficult transition a mammal is required to make in its lifetime is the change from the intrauterine environment to the extrauterine environment at birth. Every parameter of the infant's environment changes abruptly. Dramatic shifts in temperature, tactile sensation, audio stimuli, motion, and light are exacerbated by conditions in the hospital delivery room where most women in modern societies give birth. Even the environment in a loving home is alarmingly unfamiliar, and many infants exhibit prolonged crying and sleeplessness which may be related to transitional stress. It is believed that these abrupt changes in the environment may tend to intensify the infant's intrauterine to extrauterine transition and may inflict harm which affects the person's emotional and physical response to adaptive or environmental change throughout the remainder of his or her life. Therefore, a gradual and effective transition of the infant from the intrauterine environment to the extrauterine environment may have substantial long-term as well as short-term benefits.

An effective transition system would duplicate one or more aspects of the intrauterine conditions perceived by the infant just prior to birth. It would also provide means for gradually altering environmental stimuli over time until they reflect the natural extrauterine environment. Known transition systems fail to accurately simulate certain intrauterine conditions, such as the sensation of the mother's heartbeat, or the motion characteristic of the mother's gait late in pregnancy. Additionally, some known systems comprise relatively fragile suspension systems. Better solutions are needed for simulating the intrauterine environment.

### SUMMARY

The present disclosure provides systems, apparatuses, and methods relating to intrauterine simulation systems for infants.

In some embodiments, a bassinet configured to simulate an intrauterine environment comprises a movable platform configured to support a floor of the bassinet, the movable platform defining a longitudinal axis; a suspension system including: at least one linear-motion actuator attached to a fixed frame disposed underneath the movable platform and configured to linearly displace the movable platform along the longitudinal axis; and at least one rotational-motion actuator configured to impart rotational motion to the movable platform; a drive system configured to drive the suspension system; and a transducer disposed underneath the movable platform and configured to produce vibrations having frequency components preferably in any desired range of frequencies, such as approximately 5-100 Hz or approximately 20-80 Hz for bass components, up to 20,000 Hz or even ultrasonic frequencies in other embodiments.

In some embodiments, a system for transitioning an infant from an intrauterine environment comprises a low-frequency transducer coupled to an underside of a platform and configured to transmit low-frequency sound waves and/or vibrations through the platform to an upper side of the platform opposite the underside; and a motion-control system configured to translate the platform along a longitudinal axis of the platform and to rotate the platform about the longitudinal axis.

In some embodiments, an infant support system comprises a platform defining a longitudinal axis; means for translating the platform along the longitudinal axis; means for rotating the platform about the longitudinal axis; and means for inducing low-frequency acoustic waves and/or vibrations in the platform.

Features, functions, and advantages may be achieved independently in various embodiments of the present disclosure, or may be combined in yet other embodiments, further details of which can be seen with reference to the following description and drawings.

### BRIEF DESCRIPTION OF THE DRAWINGS

FIG. 1 is a set of graphs of illustrative pelvic displacements associated with a woman walking during a third trimester of pregnancy.

FIG. 2 is a graph depicting an illustrative autocorrelation function (ACF) of measured data associated with a sound of a human heartbeat.

FIG. 3 is a graph depicting a variation with time of a period associated with the autocorrelation function of FIG. 2.

FIG. 4 is a graph depicting a frequency dependence of energy associated with a left-channel measurement of the sound of a human heartbeat.

FIG. 5 is a graph depicting a frequency dependence of energy associated with a right-channel measurement of the sound of the human heartbeat.

FIG. 6 is a schematic diagram of an illustrative intrauterine simulation system, in accordance with aspects of the present teachings.

FIG. 7 is an isometric view of an illustrative bassinet incorporating an intrauterine simulation system, in accordance with aspects of the present teachings.

FIG. 8 is an exploded view of the bassinet of FIG. 7.

FIG. 9 is an isometric view of a mounting structure of the bassinet of FIG. 7 in a collapsed configuration, in accordance with aspects of the present teachings.

FIG. 10 is a side view of a portion of the bassinet of FIG. 7.

FIG. 11 is a schematic view depicting a dispersion pattern of sound waves produced by an illustrative low-frequency transducer, in accordance with aspects of the present teachings.

FIG. 12 is a plan view of an illustrative motion-control assembly, in accordance with aspects of the present teachings.

FIG. 13 is a side view of an illustrative intrauterine simulation assembly including the motion-control assembly of FIG. 12, in accordance with aspects of the present teachings.

FIG. 14 is another side view of the simulation assembly of FIG. 13.

FIG. 15 is yet another side view of the simulation assembly of FIG. 13.

FIG. 16 is a schematic diagram depicting an illustrative control system for the simulation assembly of FIG. 13.

FIG. 17 is a top view of another illustrative simulation assembly.

FIG. 18 is a side view of the simulation assembly of FIG. 17.

FIG. 19 is a sectional view of the simulation assembly of FIG. 17.

FIG. 20 is a sectional view of another illustrative bassinet including a removable core module, in accordance with aspects of the present teachings.

FIG. 21 is an isometric view of the bassinet of FIG. 20.

FIG. 22 is another sectional view of the bassinet of FIG. 20.

FIG. 23 is another isometric view of the bassinet of FIG. 20.

FIG. 24 is yet another isometric view of the bassinet of FIG. 20.

FIG. 25 is a sectional view of an illustrative cushion including an intrauterine simulation assembly, in accordance with aspects of the present teachings.

### DETAILED DESCRIPTION

Various aspects and examples of an intrauterine simulation system including motion-control and intrauterine heartbeat-simulation assemblies, as well as related methods, are described below and illustrated in the associated drawings. Unless otherwise specified, an intrauterine simulation system in accordance with the present teachings, and/or its various components, may contain at least one of the structures, components, functionalities, and/or variations described, illustrated, and/or incorporated herein. Furthermore, unless specifically excluded, the process steps, structures, components, functionalities, and/or variations described, illustrated, and/or incorporated herein in connection with the present teachings may be included in other similar devices and methods, including being interchangeable between disclosed embodiments. The following description of various examples is merely illustrative in nature and is in no way intended to limit the disclosure, its application, or uses. Additionally, the advantages provided by the examples and embodiments described below are illustrative in nature and not all examples and embodiments provide the same advantages or the same degree of advantages.

This Detailed Description includes the following sections, which follow immediately below: (1) Definitions; (2) Overview; (3) Examples, Components, and Alternatives; (4) Advantages, Features, and Benefits; and (5) Conclusion. The Examples, Components, and Alternatives section is further divided into subsections A through F, each of which is labeled accordingly.

### Definitions

The following definitions apply herein, unless otherwise indicated.

“Substantially” means to be more-or-less conforming to the particular dimension, range, shape, concept, or other aspect modified by the term, such that a feature or component need not conform exactly. For example, a “substantially cylindrical” object means that the object resembles a cylinder, but may have one or more deviations from a true cylinder.

“Comprising,” “including,” and “having” (and conjugations thereof) are used interchangeably to mean including but not necessarily limited to, and are open-ended terms not intended to exclude additional, unrecited elements or method steps.

Terms such as “first”, “second”, and “third” are used to distinguish or identify various members of a group, or the like, and are not intended to show serial or numerical limitation.

“Coupled” means connected, either permanently or releasably, whether directly or indirectly through intervening components.

“Resilient” describes a material or structure configured to respond to normal operating loads (e.g., when compressed) by deforming elastically and returning to an original shape or position when unloaded.

“Rigid” describes a material or structure configured to be stiff, non-deformable, or substantially lacking in flexibility under normal operating conditions.

“Elastic” describes a material or structure configured to spontaneously resume its former shape after being stretched or expanded.

“Processing logic” means any suitable device(s) or hardware configured to process data by performing one or more logical and/or arithmetic operations (e.g., executing coded instructions). For example, processing logic may include one or more processors (e.g., central processing units (CPUs) and/or graphics processing units (GPUs)), microprocessors, clusters of processing cores, FPGAs (field-programmable gate arrays), artificial intelligence (AI) accelerators, digital signal processors (DSPs), and/or any other suitable combination of logic hardware.

### Overview

In general, an intrauterine simulation system in accordance with aspects of the present teachings includes a motion-control assembly and/or a maternal heartbeat simulator. In some examples, the intrauterine simulation system is incorporated into a bassinet, cradle, mattress, cushion, carrier, bed, incubator, and/or other device suitable for supporting an infant. In some examples, the intrauterine simulation system is configured to be removably installed in such a device, so that the system may be conveniently moved from one device to another.

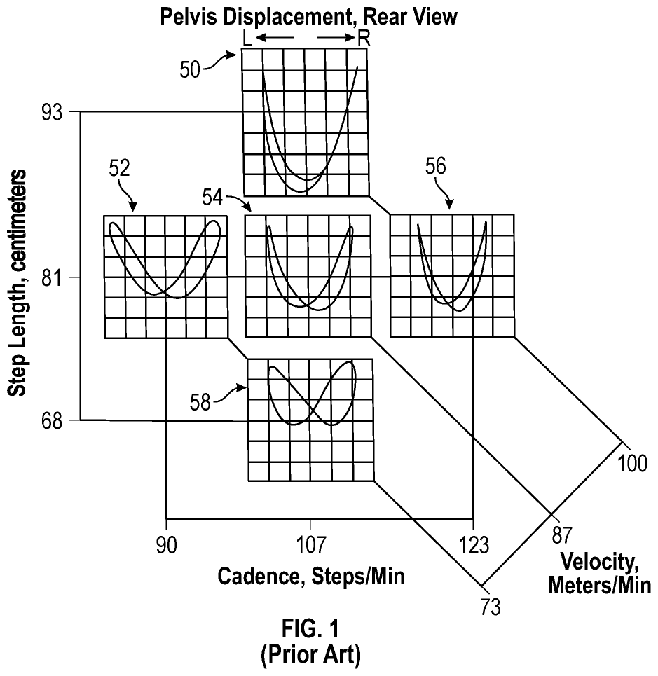

A motion-control assembly in accordance with aspects of the present teachings is configured to simulate movement experienced by a fetus prior to birth. The motion-control assembly may include, e.g., a motorized system coupled to a platform and configured to impart to the platform linear and/or rotational movement suitable for approximating movement undergone by the fetus as its mother walks. FIG. 1 depicts characteristic patterns of displacement of a pelvis of a typical pregnant woman while walking during a third trimester of pregnancy. FIG. 1 depicts a plurality of representative displacement patterns 50, 52, 54, 56, 58 from a rear view of the pelvis. The displacement occurs in both a lateral direction (e.g., left or right) and a vertical direction (up or down). As FIG. 1 shows, the displacement pattern is variable based on factors including the woman's cadence, step length, and velocity, but generally comprises a distorted and/or modified figure-eight pattern. The motion-control assembly of the present teachings is configured to approximate at least some aspects of this pattern, thereby simulating the movement of the intrauterine environment.

A maternal heartbeat simulator in accordance with aspects of the present teachings may include any suitable system configured to approximate the sensation experienced by a fetus due to the heartbeat of the mother. Unless otherwise specified, the term “heartbeat” as used herein refers generally to the activity of the maternal heart, and does not refer specifically to any particular contraction, relaxation, or other aspect of the cardiac cycle.

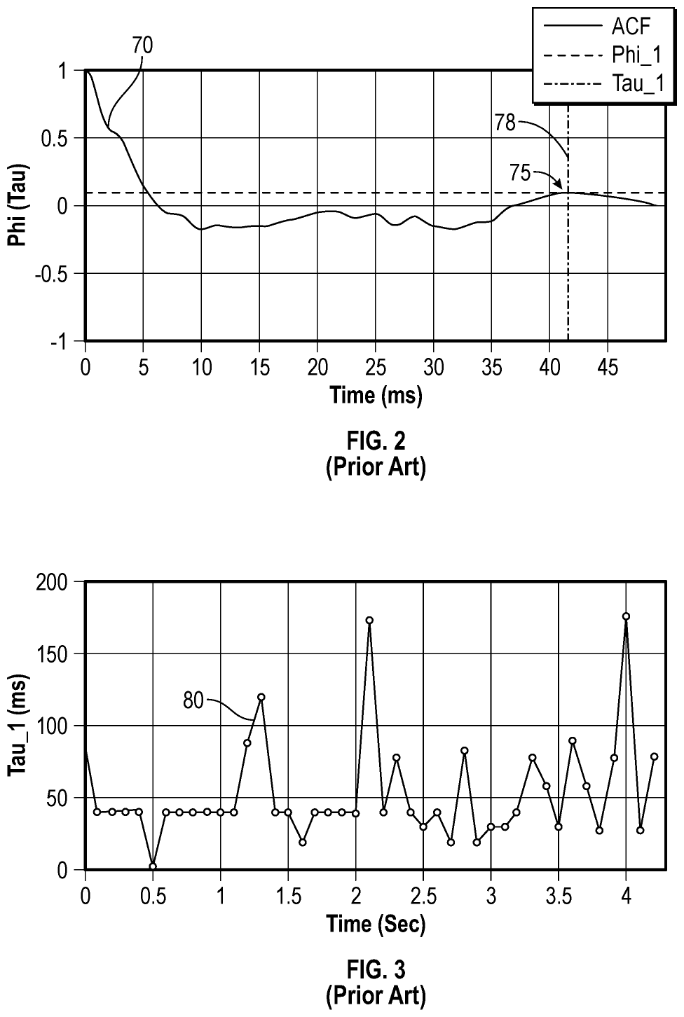

In general, a fetus in the womb experiences its mother's heartbeat as vibrations that are felt in the body rather than heard by the ear. FIGS. 2-5 depict graphs illustrating aspects of the vibrations characteristic of the human heartbeat sensation. These graphs are based on data obtained and published by Dr. Masatsugu Sakurai of Yoshimasa Electronic Inc. (see, for example, “Heartbeat measurement 1-9” at http://www.ymec.com/hp/signal2/heart1.htm—heart9.htm respectively, incorporated herein by reference).

FIG. 2 depicts data 70 associated with an autocorrelation analysis (also called an autocorrelation function, or ACF) of sounds of a human heartbeat. Data 70 represents the temporal variation of the strength (denoted Phi) of a measured signal associated with the heartbeat. Data 70 is obtained using an autocorrelation analysis rather than by direct measurement because a direct measurement of a human heartbeat (e.g., using a microphone) typically contains high levels of extraneous sound and background noise caused by the gastrointestinal tract, lungs, blood flow, and/or other systems of the body. Autocorrelation analysis is typically suitable for tasks related to the identification of a repeating pattern, such as identifying the presence of a periodic signal obscured by noise, or identifying a missing fundamental frequency based on the presence of associated harmonic frequencies. Accordingly, autocorrelation may be used to accurately determine frequency characteristics of sound produced by the human heart (e.g., by the beating of the heart). Autocorrelation enables analysis of sound qualities such as loudness, pitch, and reverberation, which may not be captured by conventional sound analysis.

As shown in FIG. 2, autocorrelation data 70 includes a peak 75 corresponding to a time of approximately 41.63 milliseconds (ms). A vertical dotted line 78 in FIG. 2 is used to indicate the 41.63 ms location on the graph. Peak 75 represents a time period (here, 41.63 ms) associated with a strong frequency component of the measured waveform. The time period is denoted by Tau_1 in the depicted graphs. The period indicated by peak 75 corresponds to the fundamental frequency of the measured signal. Here, the period at peak 75 is approximately 41.63 milliseconds, and the associated fundamental frequency is therefore 1/(0.04163 seconds), or approximately 24 Hertz (Hz).

FIG. 3 depicts a representative time-domain graph of data 80 representing a variation in time (e.g., a time course) of the period Tau_1. As the graph shows, the mean value of the period is approximately 41 ms, consistent with autocorrelation data 70. Accordingly, data 80 further indicate that a frequency of 24 Hz is characteristic of the human heartbeat.

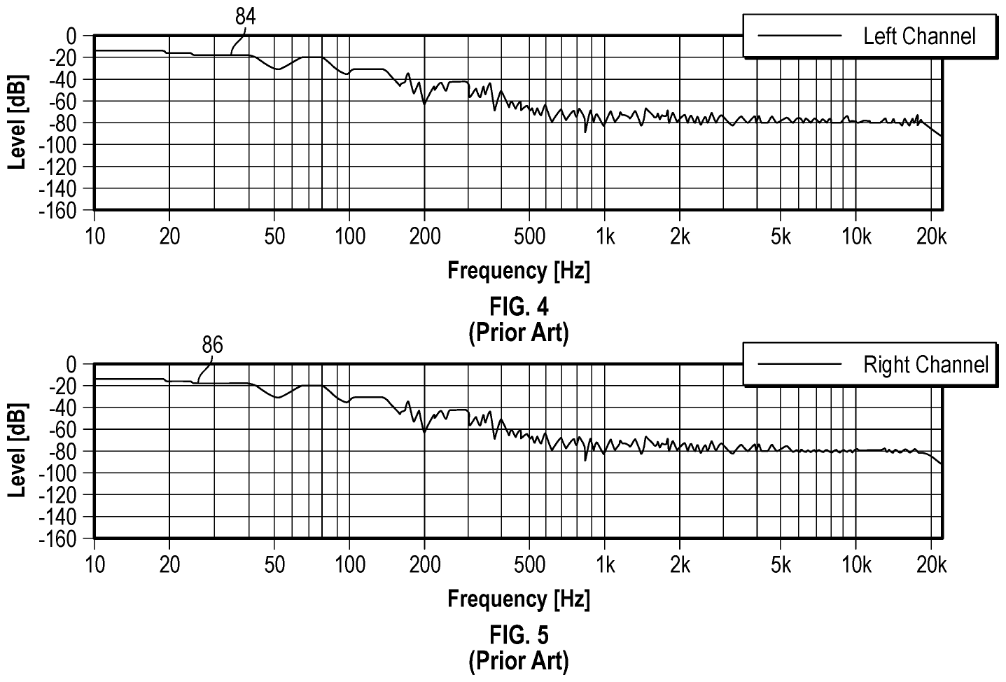

FIGS. 4-5 depict graphs of a spectrum analysis of signals measured through left and right channels of a stereo measurement device. FIG. 4 depicts left-channel data 84 representing a frequency dependence of measured energy level in decibels (dB) of the sound of the heart. FIG. 5 depicts right-channel data 86 representing the frequency-dependent energy levels measured through the right channel. Data 84, 86 show that the components of the sound spectrum having the highest energy levels are below 80 Hz. The energy levels decrease significantly in a range of approximately 100 to 600 Hz, and remain low at higher frequencies. In other words, most of the energy contained in the sound waves produced by the heart is associated with frequency components having frequencies less than 80 Hz. This is consistent with the finding illustrated by FIGS. 2-3 that the fundamental frequency component is located at 24 Hz.

Accordingly, the example data depicted in FIGS. 2-5 indicate that the measured sound of the human heartbeat has a fundamental frequency component at approximately 24 Hz, and that the majority of the aggregate energy of the sound waves is associated with frequencies under 80 Hz. Although some variation in the measured energy spectrum and/or fundamental frequencies may occur between different measurements, or different human subjects, the data presented in FIGS. 2-5 is generally representative of the low frequency of the sound produced by the heart. An accurate reproduction of the heartbeat sensation therefore includes production of low-frequency sound waves and/or vibrations (e.g., acoustic waves and vibrations having frequencies in the range of approximately 20 to 80 Hz).

Frequencies at the lower end of this range (e.g., in a range of approximately 20 to 30 Hz) are almost sub-audible and may be considered more akin to pressure waves or vibrations than to sound waves that are typically heard by the ear. In this range, sound waves are experienced in the body mainly via conduction the bones, rather than in the ear. This is consistent with the experience of the fetus in the womb, where at these frequencies, the fetus experiences the maternal heartbeat primarily through bone conduction as a full-body vibration rather than as sound waves heard in the ear.

Accordingly, the system of the present teachings is configured to generate very low-frequency vibrations that an infant can experience over a large portion of its body. In some examples, the system of the present teachings includes one or more low-frequency transducers attached to a thin, firm platform, such that vibrations generated by the low-frequency transducer(s) are transmitted to the platform and can be transmitted to an infant supported by the platform.

Suitable low-frequency transducers may include bass shakers and/or tactile transducers, or any other transducer configured to produce vibrations having suitable frequency characteristics (e.g., frequency components within or predominantly within a range of 0 to 100 Hz, 20 to 80 Hz, and/or another suitable range). As an example, Table 1 depicts respective specifications of two commercially available bass shakers sold under the names TT25-8 PUCK and TT25-16 PUCK by Dayton Audio. As the table shows, the TT25-x bass shaker has a fundamental frequency of 40 Hz and a frequency range of 20-80 Hz.

TABLE 1

| Model number | TT25-8 | | TT25-16 | |
|---|---|---|---|---|
| Power handling | 20/30 | | 20/30 | |
| (RMS/Peak) | | | | |
| Impedance | 8 | Ohm | 16 | Ohm |
| Frequency | 20-80 | Hz | 20-80 | Hz |
| response | | | | |
| Resonant | 40 | Hz | 40 | Hz |
| Frequency (Fs) | | | | |
| Force Peak | 30 | lbs/ft | 30 | lbs/ft |

| | | |
|---|---|---|
| (four-transducer | | |
| configuration) | | |
| Dimensions | 3.5 in. dia. × 1 in. H | 3.5 in. dia. × 1 in. H |
| Cut-out size | 70 mm dia. with | 70 mm dia. with |
| | 25 mm slot | 25 mm slot |

In contrast, known intrauterine heartbeat-simulation systems typically include only standard mid-range transducers, which typically produce frequencies in the range of 100 to 13,000 Hz. Furthermore, the transducer in known systems is typically disposed in a blanket, stuffed animal, bassinet rim, or other location unsuitable for transmitting low-frequency vibrations to the infant's body. Accordingly, the present system provides a more accurate simulation of the intrauterine heartbeat than those of known systems.

In some examples, the motion-control and/or heartbeat-simulation assemblies of the present disclosure are controlled by an electronic controller (e.g., processing logic). For example, the controller may drive the transducer(s) of the heartbeat-simulation system and/or a motor of the motion-control system.

In some examples, the controller is configured to vary certain aspects of the motion-control and/or heartbeat-simulation systems as a function of time. For example, the controller may be configured to sense a time of day (e.g., morning, afternoon, evening, night, etc.) and to vary the simulated motion and heartbeat sensations based on the sensed time. This may increase the accuracy of the intrauterine simulation. For example, the controller may reduce the amplitude of motions created by the motion-control system at night compared to the amplitude of motions during the day. This would be consistent with the intrauterine experience of a fetus gestated by a woman who typically sleeps at night and is awake during the day. Similarly, the pace of a simulated heartbeat may be slower during the night than during the day, and/or may simulate a REM cycle of a sleeping woman. In some examples, the controller may be switchable from a night setting to a day setting based on user input, rather than automatically based on a sensed time.

Additionally, or alternatively, the controller may be configured to gradually reduce aspects of the intrauterine simulation (e.g., amplitude of motion and/or intensity of simulated heartbeat vibrations) on a scale of weeks or months. This may help to gradually acclimate the infant to the extrauterine environment.

In some examples, one or more settings and/or functions of the controller may be modified using a remote control, a software application (e.g., a smartphone app), and/or the like.

Aspects of an intrauterine simulation system (e.g., one or more electronic controllers and/or software programs) may be embodied as a computer method, computer system, or computer program product. Accordingly, aspects of the intrauterine simulation system may take the form of an entirely hardware embodiment, an entirely software embodiment (including firmware, resident software, micro-code, and the like), or an embodiment combining software and hardware aspects, all of which may generally be referred to herein as a “circuit,” “module,” or “system.” Furthermore, aspects of the intrauterine simulation system may take the form of a computer program product embodied in a computer-readable medium (or media) having computer-readable program code/instructions embodied thereon.

Any combination of computer-readable media may be utilized. Computer-readable media can be a computer-readable signal medium and/or a computer-readable storage medium. A computer-readable storage medium may include an electronic, magnetic, optical, electromagnetic, infrared, and/or semiconductor system, apparatus, or device, or any suitable combination of these. More specific examples of a computer-readable storage medium may include the following: an electrical connection having one or more wires, a portable computer diskette, a hard disk, an SD card, a USB drive, network storage, cloud storage, a random access memory (RAM), a read-only memory (ROM), an erasable programmable read-only memory (EPROM or Flash memory), an optical fiber, a portable compact disc read-only memory (CD-ROM), an optical storage device, a magnetic storage device, and/or any suitable combination of these and/or the like. In the context of this disclosure, a computer-readable storage medium may include any suitable non-transitory, tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, and/or any suitable combination thereof. A computer-readable signal medium may include any computer-readable medium that is not a computer-readable storage medium and that is capable of communicating, propagating, or transporting a program for use by or in connection with an instruction execution system, apparatus, or device.

Program code embodied on a computer-readable medium may be transmitted using any appropriate medium, including but not limited to wireless, wireline, optical fiber cable, RF, and/or the like, and/or any suitable combination of these.

Computer program code for carrying out operations for aspects of intrauterine simulation may be written in one or any combination of programming languages, including an object-oriented programming language (such as Java, C++), conventional procedural programming languages (such as C), scripting languages (such as Python and Perl) and functional programming languages (such as Haskell). Mobile apps may be developed using any suitable language, including those previously mentioned, as well as Objective-C, Swift, C#, HTML5, and the like. The program code may execute entirely on a user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), and/or the connection may be made to an external computer (for example, through the Internet using an Internet Service Provider).

Aspects of the intrauterine simulation system may be described below with reference to flowchart illustrations and/or block diagrams of methods, apparatuses, systems, and/or computer program products. Each block and/or combination of blocks in a flowchart and/or block diagram may be implemented by computer program instructions. The computer program instructions may be programmed into or otherwise provided to processing logic (e.g., a processor of a general purpose computer, special purpose computer, field programmable gate array (FPGA), or other programmable data processing apparatus) to produce a machine, such that the (e.g., machine-readable) instructions, which execute via the processing logic, create means for implementing the functions/acts specified in the flowchart and/or block diagram block(s).

Additionally or alternatively, these computer program instructions may be stored in a computer-readable medium that can direct processing logic and/or any other suitable device to function in a particular manner, such that the instructions stored in the computer-readable medium produce an article of manufacture including instructions which implement the function/act specified in the flowchart and/or block diagram block(s).

The computer program instructions can also be loaded onto processing logic and/or any other suitable device to cause a series of operational steps to be performed on the device to produce a computer-implemented process such that the executed instructions provide processes for implementing the functions/acts specified in the flowchart and/or block diagram block(s).

Any flowchart and/or block diagram in the drawings is intended to illustrate the architecture, functionality, and/or operation of possible implementations of systems, methods, and computer program products according to aspects of the intrauterine simulation system. In this regard, each block may represent a module, segment, or portion of code, which comprises one or more executable instructions for implementing the specified logical function(s). In some implementations, the functions noted in the block may occur out of the order noted in the drawings. For example, two blocks shown in succession may, in fact, be executed substantially concurrently, or the blocks may sometimes be executed in the reverse order, depending upon the functionality involved. Each block and/or combination of blocks may be implemented by special purpose hardware-based systems (or combinations of special purpose hardware and computer instructions) that perform the specified functions or acts.

While the overview above and the examples below focus upon simulating an intrauterine environment for a human infant, the present teachings also may be used for other mammal species. For example, puppy and kitten litters may benefit from a simulated intrauterine environment. In that case, systems according to the present teachings might include alternative (and in some examples, more complex) motions, sounds, and/or vibrations, based on simulating four-legged motions of the mother as well as sounds from multiple other siblings in the womb. To some extent, such motions and sounds would also occur outside the womb after the litter is born. Similarly, the present teachings can be used to simulate the intrauterine and/or natural infant environment of any animal species, with beneficial uses for pet owners, breeders, veterinarian clients, and/or zoos, among others.

Furthermore, the present teachings extend to multiple human births (e.g., twins, triplets, and so forth), in which case systems according to the present teachings may be configured to simulate the resulting more complicated intrauterine environments. For example, the present teachings may include systems that accommodate more than one human infant, and/or that simulate the additional intrauterine movements, sounds, and vibrations associated with a multiple pregnancy.

### Examples, Components, and Alternatives

The following sections describe selected aspects of exemplary intrauterine simulation systems as well as related systems and/or methods. The examples in these sections are intended for illustration and should not be interpreted as limiting the scope of the present disclosure. Each section may include one or more distinct embodiments or examples, and/or contextual or related information, function, and/or structure.

### A. Illustrative Intrauterine Simulation System

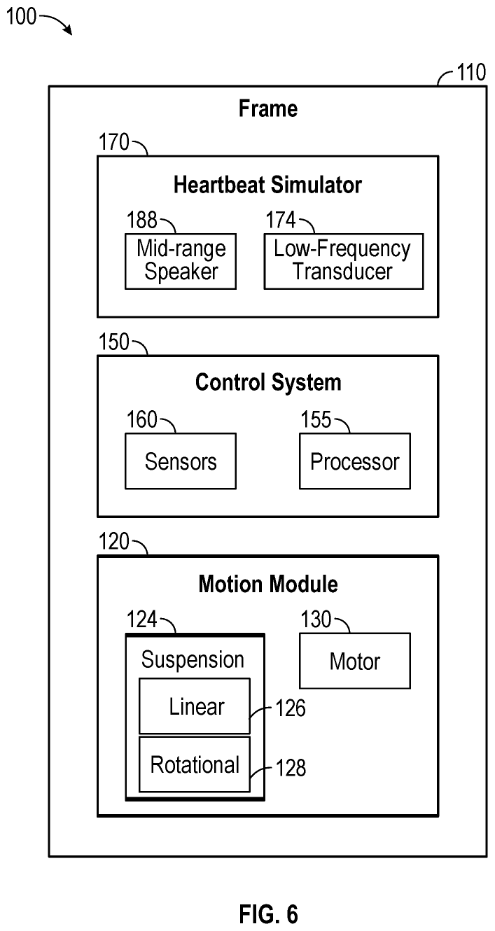

With reference to FIG. 6, this section describes an illustrative intrauterine simulation system 100, which is an example of the intrauterine simulation systems described above.

System 100 includes a frame 110. Frame 110 includes at least a platform configured to directly or indirectly support an infant. For example, frame 110 may include a thin, substantially rigid platform configured to support bedding for an infant. Frame 110 may further include a bassinet, cradle, or other suitable structure to which the platform is movably attached (e.g., such that the platform can be rotated and/or translated relative to the bassinet structure).

System 100 further includes a motion module 120, which is an example of the motion-control assembly described above. Motion module 120 may comprise any suitable mechanical components configured to impart movement to the movable platform of frame 110. Typically, motion module 120 includes a suspension and drive system 124 having a linear-motion actuator 126 configured to linearly displace the movable platform relative to the fixed frame, and a rotational-motion actuator 128 configured to rotate the movable platform relative to the fixed frame. In some examples, linear-motion actuator 126 and rotational-motion actuator 128 are coupled together (e.g., driven by a common motor and/or common belts and pulleys) to produce the complex figure-eight motion characteristic of third-trimester pelvic movement while walking. In other examples, the linear and rotational actuators are decoupled from each other to produce the figure-eight motion.

Suspension and drive system 124 may include any suitable assembly of linear and/or rotary actuators, belts and pulleys, chains and gears, and/or any other suitable devices.

Motion module 120 further includes a motor 130 configured to drive suspension and drive system 124. In some examples, motor 130 comprises a DC motor, such as a permanent-magnet type motor. Alternatively, or additionally, motor 130 may comprise a shunt motor, compound motor, and/or series motor. In some examples, motor 130 comprises an AC motor. Typically, motor 130 is suitable for quiet and smooth operation, so that operation of the motor tends not to disturb an infant sleeping in frame 110.

System 100 further includes a control system 150. Control system 150 includes one or more processors 155, which may comprise a computer, microprocessor, and/or any other processing-logic device. Processors 155 may be configured to control motor 130 (e.g., a speed of the motor). Additionally, or alternatively, processors 155 may be configured to control one or more transducers, described below.

Control system 150 may include a plurality of sensors 160. In some examples, sensors 160 include sensors configured to obtain data facilitating control of motor 130 by processor 155. For example, sensors 160 may include one or more encoders and/or speed sensors attached to motor 130, which may be used in a feedback algorithm implemented by processor 155 to maintain a speed of the motor at a target speed. Sensors 160 may further include sensors configured to obtain safety-related information, such as current sensors (e.g., for identifying excessive current in the event of a system malfunction), temperature sensors (e.g., for identifying overheating malfunctioning components), load cells (e.g., for detecting an unexpected weight distribution on frame 110), microphones (e.g., for detecting an infant's cry), and/or the like. Control system 150 may further be configured to display and/or communicate information related to the sensed information. For example, control system 150 may include one or more visible and/or audible alarms configured to be activated in response to detection of information indicating a malfunction. Additionally, or alternatively, an alert may be transmitted to an external device (e.g., a smartphone, a dedicated receiver, etc.).

System 100 further includes a heartbeat simulator 170, which is an example of a system configured to simulate sensations of a maternal heartbeat as experienced in the intrauterine environment, as described above. Heartbeat simulator 170 includes a low-frequency transducer 174. Low-frequency transducer 174 may comprise a bass transducer, bass shaker, tactile transducer, and/or any other suitable device(s) configured to produce a low-frequency waveform suitable for simulating the maternal intrauterine heartbeat. Low-frequency transducer 174 is configured to produce low-frequency sound waves and/or vibrations (e.g., having a fundamental frequency less than approximately 50 Hz, a majority of energy in the range of approximately 20 to 80 Hz, and/or any other suitable low-frequency sound waves and vibrations).

Optionally, heartbeat simulator 170 further comprises a standard mid-range transducer 188. Mid-range transducer 188 may be configured to produce higher-frequency sound waves (e.g., predominantly above approximately 100 Hz, in a range of approximately 100 Hz to 10,000 Hz, and/or any other suitable mid-frequency range). These higher-frequency sound waves may augment the low-frequency waves produced by low-frequency transducer 174 in order to produce a desired spectrum of sound and/or vibrational waves. Alternatively, or additionally, transducer 188 may be coupled to a smartphone or other communications device to play music, a human voice, an alarm, and/or any other suitable sound.

Low-frequency transducer 174 and/or transducer 188 are controlled by control system 150, which may include one or more amplifiers coupled to the transducers.

Processor 155 and/or any other suitable component of control system 150 may be configured to receive data from and/or send data to a remote control, smart phone, personal computer, laptop, or other suitable device. Data received by the controller may comprise instructions to be implemented by the controller for controlling motion module 120, heartbeat simulator 170, and/or any other suitable component of system 100. For example, processor 155 may be configured to receive instructions relating to a target speed of motor 130, a volume and/or frequency of low-frequency transducer 174, etc. Data sent by the controller may include current operating state, sensor data, and/or other information related to the status or performance of the system.

### B. Illustrative Bassinet

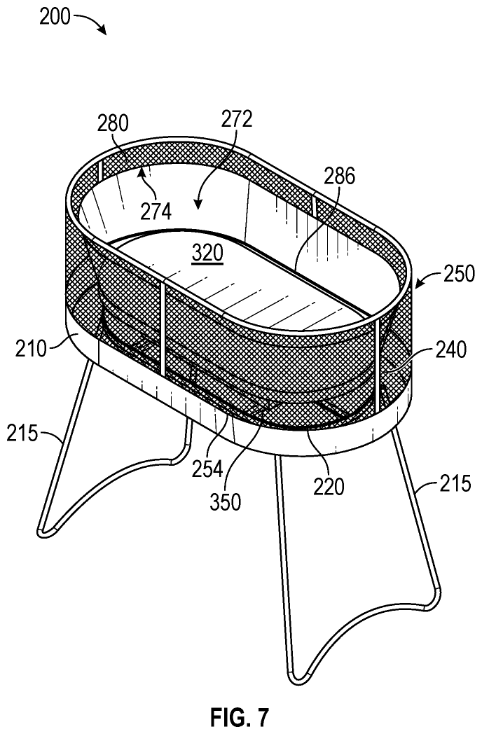

As shown in FIGS. 7-11, this section describes an illustrative bassinet 200, in accordance with aspects of the present teachings. Bassinet 200 is an example of a system configured to simulate an intrauterine environment, described above.

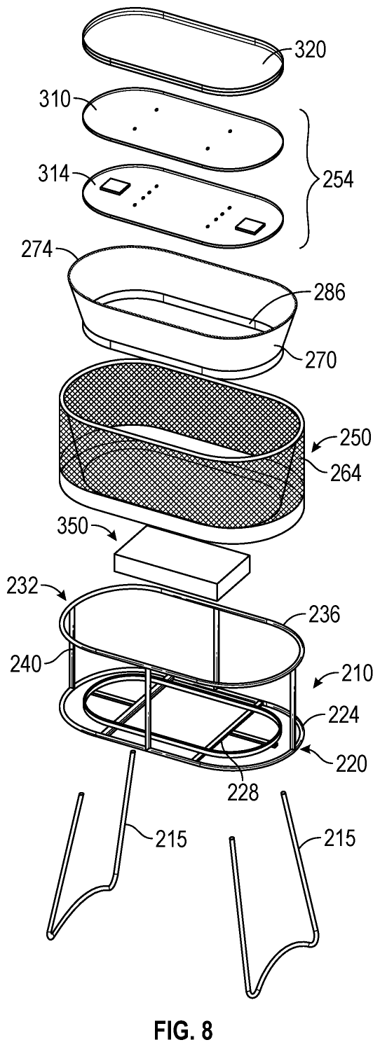

FIG. 7 is an isometric view of bassinet 200, and FIG. 8 is an exploded view of the bassinet. As FIGS. 7-8 show, bassinet 200 includes a base 210 supported by legs 215. In the depicted example, base 210 is supported above the ground by a pair of legs 215, but in other examples, the base may be supported by any suitable structure. Legs 215 may be adjustable to support base 210 at a selectable height above the ground. In some cases, the legs also may be independently adjustable, to allow the bassinet to be angled to a desired degree. For example, this may allow an infant to recline with its head slightly elevated, to address infant reflux.

Base 210 has a horizontal portion 220, which includes a base rim 224 extending around a perimeter of the horizontal portion and a plurality of base bars 228 supported by the base rim. In other examples, horizontal portion 220 may comprise a single planar structure rather than a rim and a plurality of base bars. However, the rim and base bars may provide improved air flow relative to a single plane. Additionally, or alternatively, the rim and base bars may attenuate low-frequency vibrations less than a planar base would, and accordingly may be better suited for use with a heartbeat simulation system.

Base 210 further includes a mounting structure 232 extending substantially transversely from horizontal portion 220. Mounting structure 232 has an upper rim 236 supported above base 210 by a plurality of support columns 240. Upper rim 236 is configured to support a mesh framework 250, and the mesh framework is configured to support a platform 254 above horizontal base portion 220.

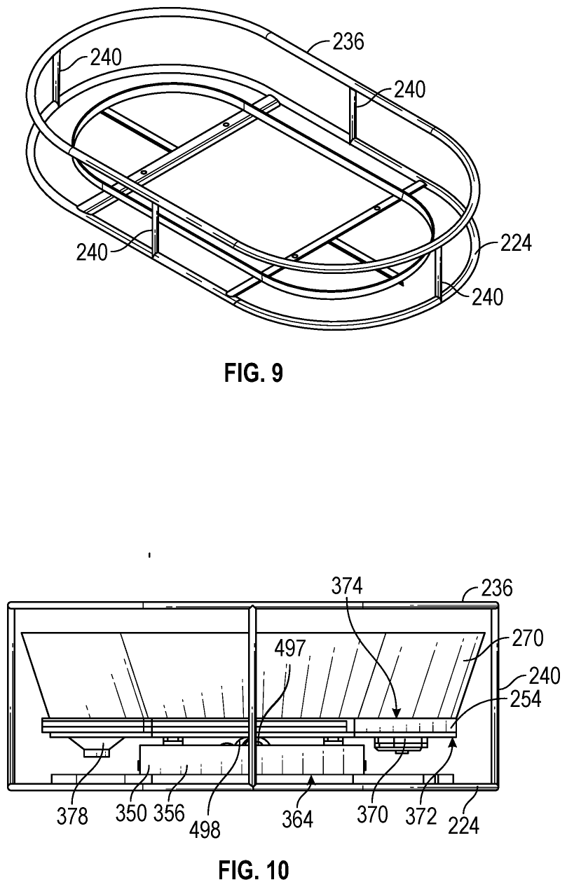

As shown in FIG. 9, support columns 240 may be collapsible, allowing base 210 to be transitionable to a more compact collapsed configuration. The collapsed configuration may be convenient for storing and/or transporting the bassinet. Additionally, or alternatively, the bassinet may be used in the collapsed configuration with an infant inside the bassinet. This may be convenient because the sides of the bassinet are shorter in the collapsed configuration, such that an infant inside the bassinet may be easier to reach. In this case, the shortened sides of the bassinet still may have a height sufficient to satisfy regulatory safety requirements, such as the requirements imposed by the U.S. Consumer Product Safety Commission.

Returning to FIGS. 7-8, mesh framework 250 may be configured to support platform 254 above the motion-control module in any suitable manner. In the depicted example, mesh framework 250 includes a substantially rigid outer wall 264 coupled to an elastic inner wall 270. Outer wall 264 is coupled to base rim 224 and to upper rim 236 of base 210 and forms a wall extending between the base rim and the upper rim. A partially enclosed interior 272 is defined by outer wall 264. In the depicted example, outer wall 264 comprises a substantially rigid mesh fabric, but in other examples the outer wall may comprise any other suitable material, or may be omitted.

Elastic inner wall 270 is disposed within interior 272, and may be coupled to outer wall 264 in any suitable manner. In the depicted example, an upper edge 274 of elastic inner wall 270 is rigidly connected (e.g., via stitches, adhesive, and/or any other suitable fasteners) to an inner portion 280 of outer wall 264 which extends into interior 272. Accordingly, elastic inner wall 270 is suspended from upper rim 236 by inner portion 280 of outer wall 264. In other examples, elastic inner wall 270 may be coupled to outer wall 264 and/or to base 210 in any other suitable way. In some examples, elastic inner wall 270 and outer wall 264 are formed by a same piece of fabric.

A lower end 286 of elastic inner wall 270 is rigidly attached to platform 254 (e.g., by adhesives, stitches, staples, nails, clamps, and/or any other suitable fasteners). In the depicted example, platform 254 comprises a top plate 310 attached to a bottom plate 314, but in other examples, the platform may comprise a single plate, or more than two plates. In some examples, elastic inner wall 270 is attached to platform 254 by clamping lower end 286 of the elastic inner wall between top and bottom plates 310, 314. The elasticity of inner wall 270 allows platform 254 to be rotated and/or translated relative to base 210.

Platform 254 is configured to support an infant and to propagate low-frequency sound waves and/or vibrations (e.g., sound waves and vibrations having frequencies less than approximately 100 Hz). For example, platform 254 may be thin and firm.

A mattress 320 may be disposed on platform 254 within interior 272. Mattress 320 is optional, and may improve the comfort of bassinet 200 for an infant disposed in the bassinet.

A simulation module 350 is disposed on horizontal portion 220 of base 210. As described below, simulation module 350 includes components configured to perform motion-control and/or heartbeat-generation functions to simulate an intrauterine environment.

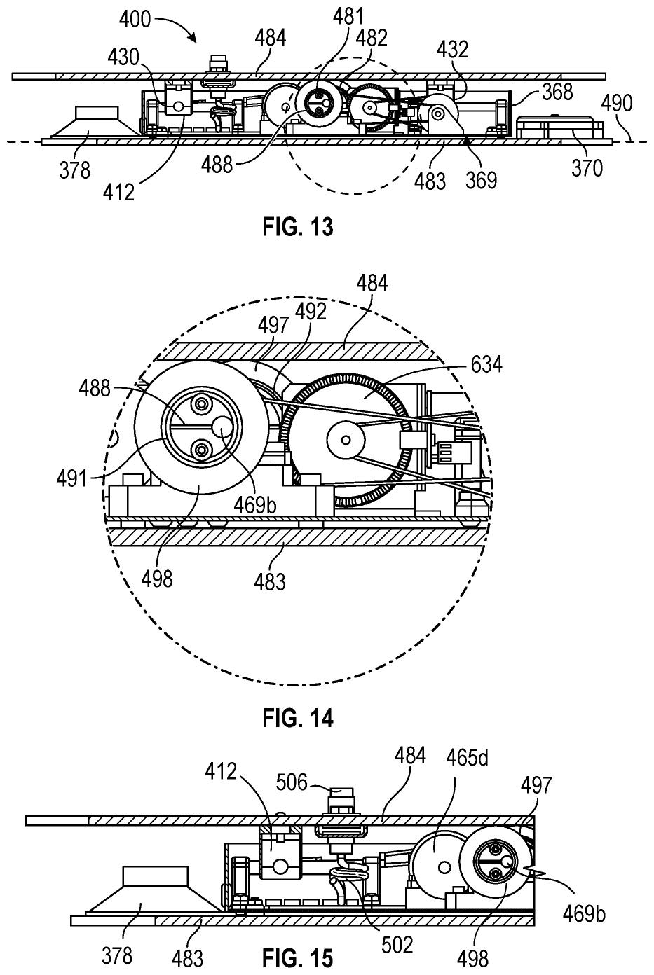

FIG. 10 is a side view depicting elastic inner wall 270 and platform 254 within base 210. Outer wall 264 is omitted from FIG. 10 for clarity. As FIG. 10 shows, simulation module 350 is disposed between horizontal base portion 220 and platform 254. In the example depicted in FIG. 10, simulation module 350 includes a box 356 having a box floor 364. Box 356 is open at the top, and box floor 364 is attached to horizontal base portion 220 by one or more bolts, screws, nails, staples, adhesives, and/or any other suitable fasteners. In other examples (see, e.g., FIGS. 13-15), simulation module 350 instead includes a box 368 that has a box top 369 and is open at the bottom. In either case, simulation module 350 includes a motion-control assembly configured to simulate movement of the intrauterine environment (e.g., during a third trimester of gestation). An example motion-control assembly is described below.

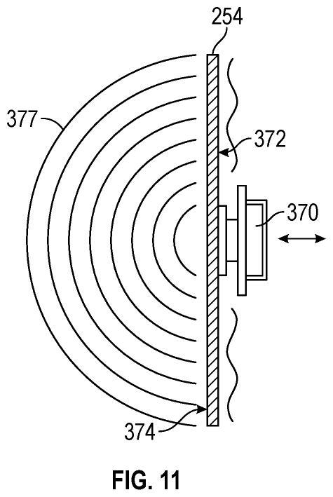

A bass shaker 370 is attached to a bottom side 372 of platform 254. Bass shaker 370 is an example of low-frequency transducer 174, described above. Accordingly, bass shaker 370 is configured to produce low-frequency sound waves and/or vibrations that propagate through platform 254 (e.g., from bottom side 372 toward an opposing top side 374, to an infant supported on the top side of the platform). FIG. 11 depicts an illustrative sound wave and/or vibration propagation pattern 377 (also referred to as a planar sound wave dispersion pattern) that may be suitable for approximating the intrauterine heartbeat.

Optionally, a mid-range transducer 378 is also attached to bottom side 372. Transducer 378 is an example of mid-range transducer 188, described above.

### C. Illustrative Simulation Assembly

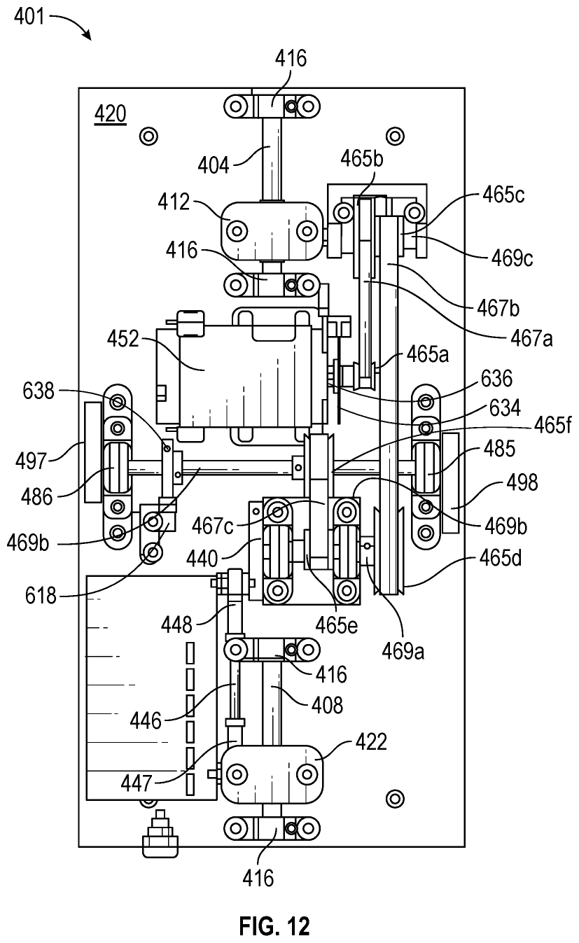

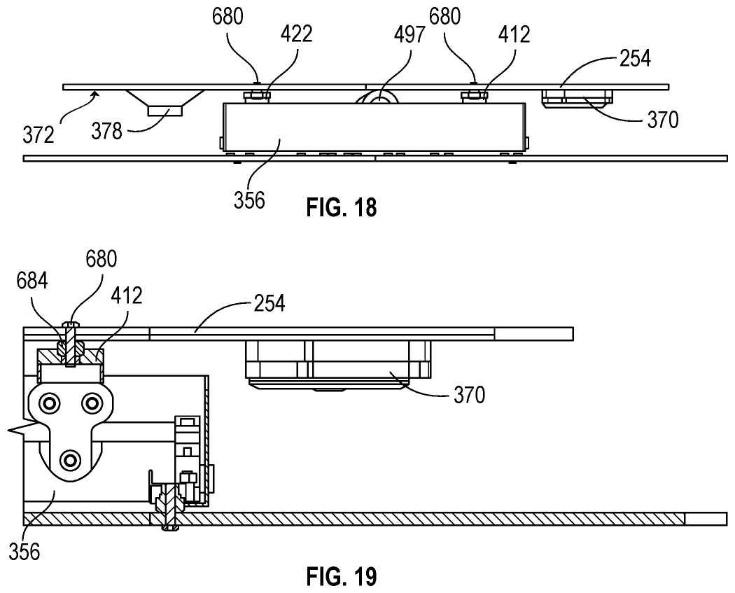

With reference to FIGS. 12-19, this section describes an illustrative simulation assembly 400 including a motion-control assembly 401. Simulation assembly 400 is an example of simulation module 350 suitable for use in bassinet 200, described above. Assembly 401 is an example of motion module 120, described above.

FIG. 12 is a plan view of motion-control assembly 401. Assembly 401 includes a robust suspension system comprised of first and second slide shafts 404 and 408 (also referred to as rods). First slide shaft 404 is slideably supported by a shaft support 412. A pair of rod fasteners 416 rigidly attach slide shaft 404 to a plate, indicated in FIG. 12 by the numeral 420. Depending on the embodiment, plate 420 may comprise a top or floor of a box of simulation module 350.

Second slide shaft 408 is slideably supported by a driving shaft support 422 and is rigidly attached to plate 420 by additional rod fasteners 416. Slide shafts 404, 408 are constructed from finely polished high-grade steel and/or any other material suitable for smooth motion and a long service life.

As described below, second slide shaft 408 is driven within driving shaft support 422, thereby linearly translating plate 420 (relative to another component, see below). First slide shaft 404 slides within support 412 as the plate is translated. In some examples, slide shafts 404, 408 are configured to be displaced relative to support 412 and driving support 422 respectively by a distance of approximately 1.5 inches (e.g., a displacement of approximately 0.75 inches in a first direction, and a displacement of approximately 0.75 inches in an opposing second direction). This corresponds to the average up-and-down displacement the infant experiences in the womb when the mother is walking. However, slide shafts 404, 408 may be configured for a different amount of displacement if desired. For example, a greater displacement may be used to better simulate the intrauterine environment of an infant whose mother is unusually tall. Additionally, or alternatively, the amount of displacement may be varied over time. For example, the displacement may be decreased over the weeks or months following the infant's birth, thereby acclimating the infant to the extrauterine environment.

Typically, in the womb, the infant is oriented in an approximately vertical position (e.g., with its head substantially toward or away from the ground when the mother is standing), but after being born the infant typically sleeps and rests in a horizontal position. Accordingly, assembly 401 is typically oriented (e.g., within bassinet 200 or other suitable structure) such that slide shafts 404, 408 move in a horizontal plane (e.g., a plane substantially coplanar with the ground during normal use).

First slide shaft 404 is slideably supported within shaft support 412 by at least one bushing 430, and second slide shaft 408 is slideably supported within driving shaft support 422 by at least one bushing 432 (see FIG. 13). Bushings 430, 432 enable shafts 404, 408 to rotate within respective supports 412, 422. Rotational motion of assembly 401 is described below.

Driving shaft support 422 is operatively coupled to a cam pulley 440 via a driving rod 446 having rod end linkages 447, 448. A motor 452 is configured to drive cam pulley 440 via a plurality of pulleys 465a-465e, belts 467a-467c, and shafts 469a-469c. These pulleys and belts can be configured in various ratios to provide different levels of torque and RPM depending on the motor type and size. The number and arrangement of pulleys, belts, and shafts in FIG. 12 is an illustrative example, and any suitable assembly for driving second slide shaft 408 and/or first slide shaft 404 may be used.

Assembly 401 further includes a pair of offset cams 481, 482 (see FIG. 13) configured to cause rotational motion. Depending on the installation of simulation module 350, offset cams 481, 482 may either impart rotational motion to plate 420, or may rotate another platform relative to plate 420. FIGS. 13-15 depict an example wherein assembly 401 is disposed substantially within open-bottomed box 368. Box top 369 is an example of plate 420 indicated in FIG. 12. Box top 369 is disposed adjacent and/or engaging a top plate 483. Offset cams 481, 482 protrude from the open bottom of the box to push a bottom plate 484. Shaft support 412 and driving shaft support 422 are rigidly attached to bottom plate 484. Typically the infant would be supported above top plate 483.

Offset cams 481, 482 are attached to shaft 469b. Shaft 469b rotates on a pair of bearing pillow blocks 485, 486 and is driven by pulley 465f and belt 467c, which is powered from pulley 465e. Bearing pillow blocks 485, 486 are rigidly attached to box top 369. Additionally, or alternatively, the bearing pillow blocks may be rigidly attached to top plate 483.

Offset cams 481, 482 each have a lobe 488 offset from a center of shaft 469b by a predetermined distance. The predetermined distance may be within a range of 0 to 15 millimeters (mm), 5 to 10 mm, and/or any other suitable range. In the depicted example, the distance is approximately 7.8 mm, which corresponds to an angular displacement of +/−4.5 degrees rotation. Offset cams 481, 482 are mounted on shaft 469b, which is oriented substantially transverse to a longitudinal axis 490 defined by plate 483. Offset cams 481, 482 are offset from each other by 180 degrees, such that when one of the cams is at its highest position, the other cam is at its lowest position. Accordingly, rotating the cams together (e.g., by rotating shaft 469b) causes plate 483 to rotate about longitudinal axis 490 defined by the plate.

Each of offset cams 481, 482 has a respective ball bearing 491, 492 pressed over its outer surface. A respective tire 497, 498 made from a resilient plastic or rubber material, and/or the like, is press-fitted over each ball bearing 491, 492. Mechanical linkages of assembly 401 are designed such that both tires 497, 498 have a slight pressure on the bottom plate 484 and are in continuous contact with it. The arrangement of two offset cams, one on each side of longitudinal axis 490, allows a very smooth rocking action.

The ratio between the longitudinal motion cycle and the rotational motion cycle is 2:1. In other words, for each full cycle of cam pulley 440, the platform only rotates a half-cycle. This 2:1 ratio is based on the motion of the mother's hips (see FIG. 1). Typically, assembly 401 does not allow the ratio to be varied, but in some examples, the ratio may be variable. The ratio is determined by pulleys 465e and 465f.

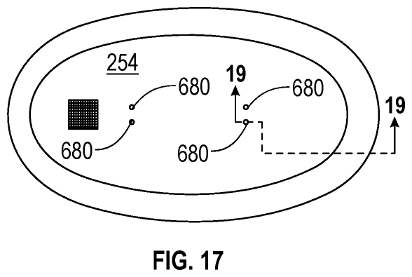

Alternatively, as described above, assembly 401 may be disposed within open-top box 356. FIG. 10, described above, depicts such an example, as do FIGS. 17-19, described below. In the example depicted in FIG. 10, floor 364 of box 356 is rigidly attached to horizontal base portion 220, and offset cams 481, 482 (e.g., tires 497, 498 attached to the cams) extend from the open top of the box to push against platform 254. The offset of the cams causes platform 254 to rotate relative to horizontal base portion 220. Pillow bearing blocks 485, 486 are rigidly attached to box floor 364 and/or to horizontal base portion 220. The sliding shafts and associated supports may be configured in any manner suitable to enable motion of platform 254 relative to base 210. For example, shaft supports 412, 422 may be rigidly attached to box floor 364 and/or to horizontal base portion 220, with slide shafts 404 and 408 rigidly attached to bottom side 372 of platform 254.

FIG. 15 depicts an interior power cable 502 within box 368 and configured to supply power to motor 452 and/or any other suitable components of assembly 400 (e.g., an electronic controller, bass shaker 370, mid-range transducer 378, one or more sensors, etc.). Interior power cable 502 is configured to be coupled to an external power cable by a power connector 506.

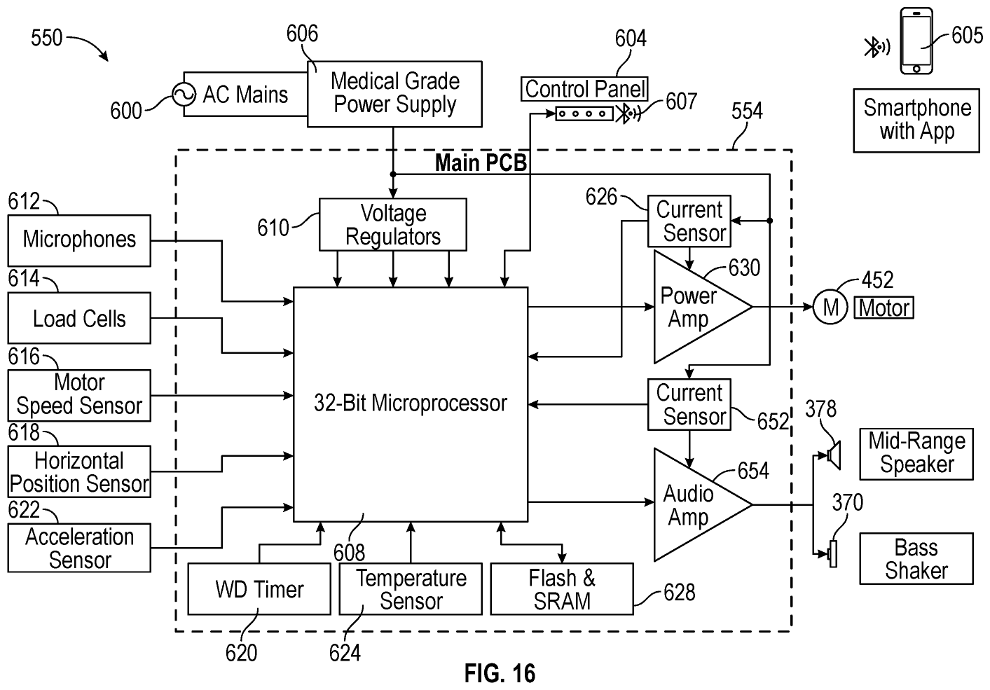

FIG. 16 schematically depicts an illustrative control system 550 in accordance with aspects of the present teachings. Control system 550 is an example of a control system 150, described above.

Electrical power is delivered to bassinet 200 (or another suitable device) through control system 550. An external power source, such as an AC mains power 600, is coupled to a medical-grade low-voltage DC power supply 606 (e.g., through power cable 502 and connector 506, described above with reference to FIG. 15).

A control panel 604 disposed on or adjacent power supply 606 provides a user interface configured to allow a user to turn the power supply to the bassinet on or off, to start or stop motor 452, and/or to change other settings of the bassinet (based on a time of day or night, an age of the infant, etc.). In some examples, control panel 604 is a backup control panel intended to be used primarily when a main controller (such as a smartphone 605, remote control, computer, or other suitable device) is unavailable.

Control panel 604 may be disposed on any suitable location of bassinet 200 (or another suitable device. For example, control panel 604 may be disposed on base 210 of bassinet 200.

Control system 550 is configured to receive and/or transmit communications (e.g., to smartphone 605) via a Bluetooth wireless communication system 607. In some examples, communications may additionally or alternatively be made via a dedicated wired communications connection.

In some examples, power supply 606 and wireless communication system 607 are located at a distance from interior 272 of bassinet 200, thereby reducing potential and/or perceived hazards from high voltages, electromagnetic fields, and/or high-frequency radiated energy.

Control system 550 includes a main printed circuit board assembly 554 (also called a PCB or PCBA) including a processor 608. In the depicted example, processor 608 comprises a 32-bit microprocessor, but in other examples, any suitable processor or processors may be used. One or more voltage regulators 610 regulate the voltage supplied to processor 608 by power supply 606.

A plurality of sensors are coupled to processor 608. In the depicted example, sensors coupled to processor 608 include a microphone 612, load cell(s) 614, motor speed sensor 616, horizontal position sensor 618, watchdog timer 620, acceleration sensor 622, temperature sensor 624, and current sensor 626.

Current sensor 626 in combination with acceleration sensor 622 is monitored and logged to provide real-time safety. If motor 452 malfunctions and produces excess current, or if the entire platform experiences an unexpected or sudden movement (e.g., acceleration), sensors 622, 626 signal processor 608 to quickly halt the motor drive. For example, a dangerous situation could occur if the infant's toddler sibling climbed onto bassinet 200 while motor 452 is operating, and sensors 622, 626 could detect this situation and signal processor 608 to stop the motor. In some examples, sensors 622, 626 are configured to log any abnormal activity, and/or to periodically log normal activity (e.g., in a flash and/or SRAM memory store 628). Logged data relating to normal and/or abnormal activity can be used to predict equipment failures and help guide preventative maintenance.

Microphone(s) 612 is configured to monitor sounds in and/or adjacent bassinet 200 (e.g., sounds made by an infant within the bassinet). Processor 608 is configured to analyze sounds received by microphone 612 (e.g., in real-time) to detect occurrences of crying, cooing, and/or the like. In response to detecting crying (and/or another type of sound), a record may be made in memory store 628. In some examples, the record comprises an indication that crying occurred, and may further comprise data associated with the time at which the crying was recorded. Additionally, or alternatively, an audio recording of the actual detected sound may be stored. In some examples, microphone 612 is omitted.

Load cells 614 enable weighing of the infant. Processor 608 is configured to monitor outputs from the load cells and to compare output corresponding to an empty platform to output corresponding to the presence of an infant to determine the infant's weight. The weight is logged (e.g., in memory 628). Memory 628 may be accessible via smartphone 605 and/or any other suitable device in communication with the memory.

Watchdog timer 620 is configured to monitor the execution of instructions (e.g., code) by processor 608. Code correctly run by processor 608 will reset watchdog timer 620 on a regular interval. Failure of watchdog timer 620 to reset within a predetermined time period typically indicates a system error. If watchdog timer 620 is not reset in the expected amount of time, then the timer times out and causes a reboot of processor 608 and/or other components of system 550.

Temperature sensor 624 is included on main PCB 554 as a safety feature configured to detect over-temperature conditions, which are expected never to occur during normal operation. In response to detection of an excessive temperature by temperature sensor 624, processor 608 may be configured to activate an alarm, halt operation of motor 452, and/or perform any other suitable action.

Control system 550 is configured to control motor 452. A power amplifier 630 is coupled to processor 608 and to motor 452, and configured to amplify a signal from the processor to control the speed (e.g., RPM) of the motor. Processor 608 determines a suitable target speed for the motor based on a current mode of operation of assembly 401 (e.g., ramping up, ramping down, steady speed, night mode, day mode, etc.). The mode of operation may be settable by a user via control panel 604 and/or smartphone 605. Based on the mode of operation, processor 608 drives motor 452 at an appropriate level.

Processor 608 is coupled to an encoder disk 634 attached to a shaft 636 of motor 452 (see FIG. 12). Processor 608 is configured to receive data from encoder disk 634 representing a speed of rotation of shaft 636. Motor speed sensor 616 of control system 550 is configured to measure a time interval between pulses of encoder disk 634. Based on the data received from disk 634 and the time measured by motor speed sensor 616, processor 608 determines a speed of motor 452 and, as needed, adjusts a supply of power to the motor such that a target speed of the motor is maintained. This enables motor 452 to be operated at the target speed irrespective of changes in position of the infant, changes in weight in the bassinet, and/or any other factors.

When movement of platform 254 is stopped, it is desirable that the platform come to rest in a horizontal position (e.g., substantially level with the ground), so that the infant supported by the platform does not roll. Processor 608 is configured to stop motor 452 in response to receiving information from horizontal position sensor 618 indicating that the platform is level. Horizontal position sensor 618 may comprise any suitable sensor configured to detect whether the platform is level. In the depicted example, horizontal position sensor 618 is configured to sense a presence and/or position of a horizontal position indicator 638 (see FIG. 12) which is mounted on a circumference of cam pulley 440. Indicator 638 may comprise any device detectable by horizontal position sensor 618. For example, sensor 618 may comprise a Hall-effect sensor, and indicator 638 may comprise a magnet. Additionally, or alternatively, sensor 618 may be configured to detect indicator 638 using a simple mechanical switch, optically and/or via RFID. Yet another alternative or additional approach is to detect a horizontal position using software, following a calibration step.

Processor 608 is further configured to control bass shaker 370 and mid-range transducer 378 (if included). Typically, processor 608 is configured to drive an audio amplifier 654, which is configured to drive bass shaker 370 and optional mid-range transducer 378. Amplifier 654 amplifies audio waveforms supplied by processor 608. In some examples, respective dedicated amplifiers are configured to drive bass shaker 370 and mid-range transducer 378.

A current sensor 652 is configured to monitor current levels within audio amplifier 654. Current sensor 652 is coupled to processor 608. In response to a detection of excessive current levels within amplifier 654 by current sensor 652, processor 608 may be configured to sound an alarm, stop a power supply to the amplifier, and/or take any other suitable action. In this manner, current sensor 652 prevents bass shaker 370 and mid-range transducer 378 from exceeding a predetermined cutoff power level.

As described above, processor 608 and amplifier 654 drive bass shaker 370 at relatively low frequencies (e.g., predominantly in a range of 20 to 80 Hz), such that low-frequency vibrations propagate from bass shaker 370 through the attached platform (e.g., plate 483 and/or platform 254) in the manner depicted in FIG. 11 (e.g., in planar dispersion pattern 377). This provides an infant on the platform with a simulated substantially full-body intrauterine heartbeat experience.

To enable platform 254 to radiate low-frequency waves accurately (e.g., without significant distortion and/or attenuation), the platform is thin, firm, and configured to be isolated from non-moving masses likely to distort and/or attenuate the waves. Accordingly, one or more vibration-isolating components may be used to connect the platform to which bass shaker 370 is attached to motion-control assembly 401.

FIGS. 17-19 depict bass shaker 370 and mid-range transducer 378 attached to underside 372 of platform 254. A plurality of fasteners 680 attach shaft support 412 and driving shaft support 422 to platform 254. Each fastener 680 passes through a respective isolation grommet 684. Isolation grommet 684 vibrationally isolates platform 254 (to which the transducers are attached) from box 356 (within which motion-control assembly 401 is substantially disposed). Isolation grommets 684 may comprise any suitable material for reducing and/or substantially preventing vibrational coupling between box 356 and platform 254. For example, isolation grommets 684 may comprise a low-durometer elastomeric material.

In other examples, isolation grommets 684 may be omitted and vibration isolators comprising another form and/or material may be used. Suitable vibrationally isolating devices may include pieces of rubber, cork, foam, and/or laminate; mechanical springs or spring dampeners; tuned mass dampeners; pneumatic isolators; and/or the like.

D. Illustrative Bassinet with Removable Core Module

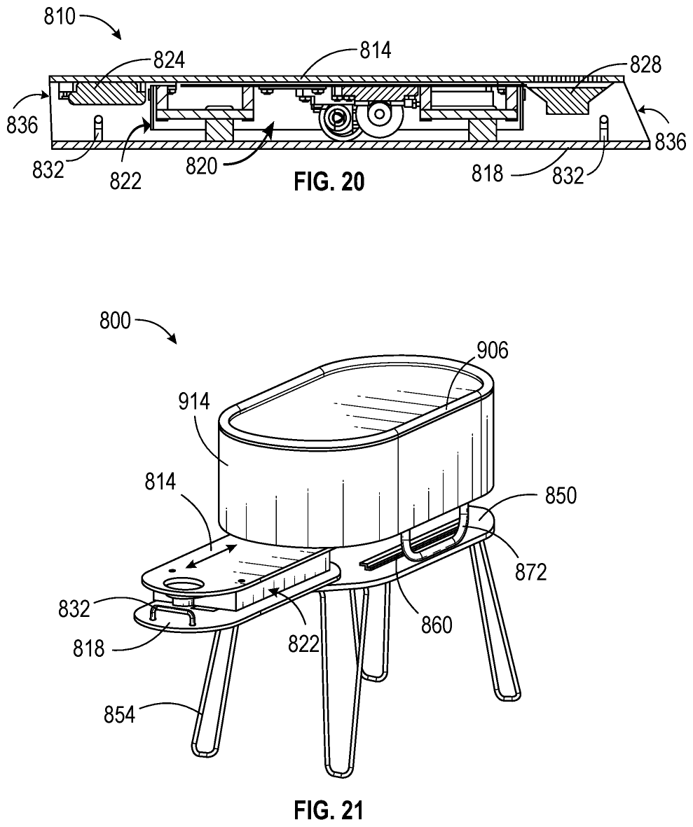

With reference to FIGS. 20-24, this section describes an illustrative bassinet 800 having a removable core module 810. Bassinet 800 is another example of a system configured to simulate an intrauterine environment, described above. Removable core module 810, as described below, includes components configured to simulate the intrauterine experience, and is configured to be readily removable from bassinet 800.

FIG. 20 is a sectional view of removable core module 810. Removable core module 810 includes a top platform 814 and a bottom platform 818. Between top and bottom platforms 814, 818 is a motion-control assembly 820, which may be substantially similar to motion-control assembly 401, described above. Assembly 820 is at least partially enclosed by an open-bottomed box 822. In other examples, assembly 820 may be partially enclosed by an open-top box.

A bass shaker 824 and, optionally, a mid-range transducer 828 are attached to top platform 814. Bottom platform 818 includes a pair of handles 832 configured to facilitate handling of removable core module 810. In other examples, handles or other suitable devices may additionally or alternatively be disposed elsewhere on module 810.

Optionally, a membrane 836 may cover all or a portion of removable core module 810. Membrane 836 may prevent debris and/or fluids from entering removable core module 810, helping to keep the module hygienic and preventing damage to the module's components. Membrane 836 may comprise any suitable flexible, stretchable, and substantially waterproof material. For example, membrane 836 may comprise rubber, latex, polychloroprene, nylon, and/or any other suitable material. Membrane 836 stretches over substantially all of top platform 814 and over at least a portion of bottom platform 818, or vice versa. Open sides of the module between top and bottom platforms 814, 818 are sealed by membrane 836.

In some examples, bassinet 800 is used with removable core module 810 for an interval of days, weeks, and/or months, facilitating the transition of one or more infants from the intrauterine environment to the extrauterine environment. After a suitable transition-time interval has elapsed, removable core module 810 may be removed from the bassinet and installed in a different bassinet or other suitable device (e.g., to help transition a different infant in a different location). In some examples, membrane 836 is discarded after being used to transition a first infant and replaced prior to being used to transition the next infant. This may increase the hygiene and/or perceived hygiene of removable core module 810.

As shown in FIG. 21, which is an isometric view, bassinet 800 includes a bassinet base 850 supported by a plurality of legs 854. Membrane 836 is omitted from FIGS. 21-24 for clarity. Lower guide rails 860 attached to base 850 facilitate sliding bottom platform 818 of removable core module 810 into position in the bassinet, and retaining the core module against the base. Similarly, upper guide rails 864 attached to a movable platform 868 (see FIG. 24) guide top platform 814 of core module 810 into position and retain the top platform against the movable platform. A spring stop 870 (see FIG. 24) engages bottom platform 818, thereby helping to retain removable core module 810 in place.

A pair of opposing supports 872 support movable platform 868 above base 850, so that the movable platform is supported above the base irrespective of whether removable core module 810 is present.

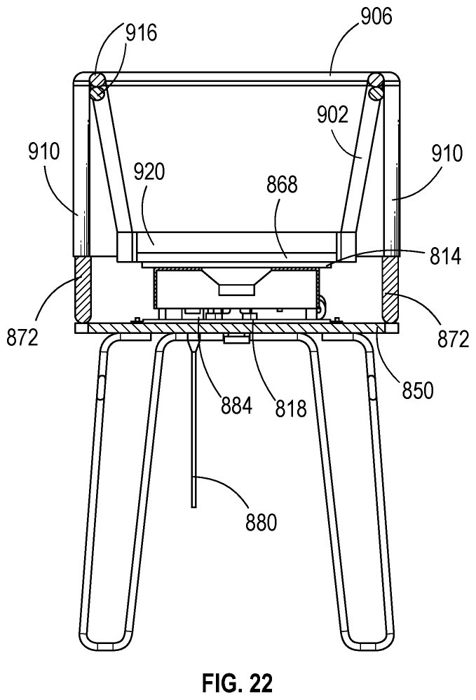

As shown in FIG. 22, which is a sectional view, a power cable 880 provides power to the bass shaker, transducer, and motion-control assembly. Power cable 880 is coupled to assembly 820 through a power bracket 884. In some examples, power bracket 884 is configured to prevent core module 810 from being removed from bassinet 800 while power cable 880 is connected to the power bracket. For example, power bracket 884 may be coupled to spring stop 870 and configured to prevent the spring stop from being pressed down to allow the core module to be removed while power cable 880 is connected to the power bracket. Accordingly, removing core module 810 from bassinet 800 includes disconnecting power cable 880 from power bracket 884, pressing spring stop 870, and pulling and/or pushing the core module out of the bassinet (e.g., using one or more handles 832).

Movable platform 868 is suspended by a stretchable fabric netting 902 from an upper rim 906. Upper rim 906 is supported by side rims 910. An outer netting 914 (see FIG. 21) extends between upper rim 906 and a bottom of side rims 910. In the depicted example, outer netting 914 and inner netting 902 comprise a single piece of fabric stretched over upper rim 906 to form the inner and outer netting. In other examples, the inner and outer netting may comprise distinct pieces of fabric. Tubing 916 may help to hold the inner and outer netting in place. Inner netting 902 allows movable platform 868 to move relative to base 850. A mattress 920 may be disposed on movable platform 868.

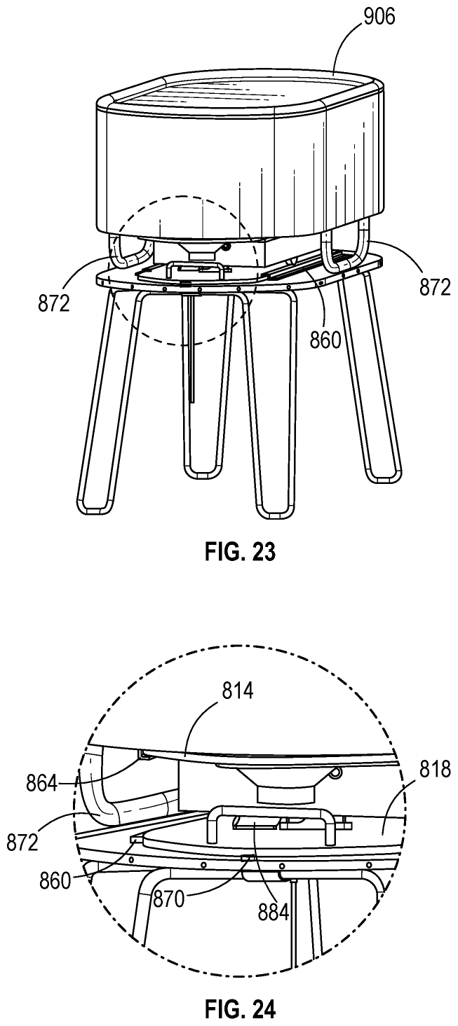

FIGS. 23-24 are additional isometric end views of an end of bassinet 800, depicting removable core module 810 disposed within the bassinet.

### E. Illustrative Cushion

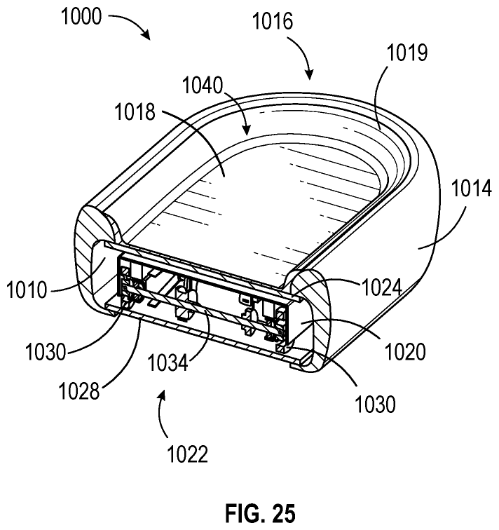

With reference to FIG. 25, this section describes an illustrative cushion 1000 configured to simulate an intrauterine environment for an infant disposed on the cushion. Cushion 1000 is another example of a system configured to simulate an intrauterine environment, described above.

FIG. 25 is an isometric cutaway view depicting cushion 1000. Cushion 1000 has an interior cavity 1010 at least partially encased in a resilient exterior 1014. Resilient exterior 1014 may comprise foam, down, microbeads, hollow silicone rubber, and/or any other material suitable for comfortably supporting an infant and allowing for longitudinal and rotation motions produced by a motion-control assembly.

An upper portion 1016 of exterior 1014 may include a depressed central portion 1018 disposed within a raised perimeter portion 1019. An infant may be placed in depressed central portion 1018, and raised portions 1019 may help to prevent the infant from rolling off of cushion 1000, or from dropping items off the side of the cushion. Additionally, or alternatively, central portion 1018 has relatively little (relative to raised portions 1019) or no cushioning underneath, and therefore may propagate low-frequency acoustic waves and/or vibrations produced by a bass transducer with relatively low attenuation. However, an infant may be placed on cushion 1000 in any suitable way.

A simulation module 1020 is disposed within cavity 1010. In the depicted example, module 1020 includes a motion-control assembly 1022 disposed between a top platform 1024 and a bottom platform 1028, and may further include a bass shaker (not shown) and/or a mid-range transducer (not shown). Motion-control assembly 1022 may be substantially similar to motion-control assembly 401, described above. For example, as shown in FIG. 25, assembly 1022 includes a pair of offset cams 1030 mounted to a shaft 1034.

Exterior 1014 encloses module 1020 for safety and aesthetic reasons. Exterior 1014 may include a substantially waterproof liner 1040 configured to prevent ingress of fluids into cavity 1010. Liner 1040 is optional and may be omitted. In some examples, exterior 1014 comprises a waterproof material (e.g., silicone rubber) even without a liner.

### F. Illustrative Combinations and Additional Examples

This section describes additional aspects and features of infant support systems configured to simulate an intrauterine environment, presented without limitation as a series of paragraphs, some or all of which may be alphanumerically designated for clarity and efficiency. Each of these paragraphs can be combined with one or more other paragraphs, and/or with disclosure from elsewhere in this application, including the materials incorporated by reference in the Cross-References, in any suitable manner. Some of the paragraphs below expressly refer to and further limit other paragraphs, providing without limitation examples of some of the suitable combinations.
A0. A bassinet configured to simulate an intrauterine environment, the bassinet comprising a movable platform configured to support a floor of the bassinet, the movable platform defining a longitudinal axis; a suspension system including: at least one linear-motion actuator attached to a fixed frame disposed underneath the movable platform and configured to linearly displace the movable platform along the longitudinal axis; and at least one rotational-motion actuator configured to impart rotational motion to the movable platform; a drive system configured to drive the suspension system; and a transducer disposed underneath the movable platform and configured to produce vibrations having frequency components in a range of approximately 20-80 Hz.
A1. The bassinet of paragraph A0, wherein the movable platform, the suspension system, and the drive system are configured to be removable from the bassinet.
A2. The bassinet of any one of paragraphs A0 through A1, wherein the drive system includes an electronic controller configured to vary the linear displacement and rotational motion based on a time of day.
A3. The bassinet of any one of paragraphs A0 through A2, wherein the at least one rotational-motion actuator of the suspension system comprises a pair of cams attached to a shaft disposed transverse to the longitudinal axis, and wherein the cams are offset from each other.
A4. The bassinet of paragraph A3, wherein the drive system is configured to drive the linear-motion actuator and the pair of cams to move the movable platform in a modified figure-eight pattern.
A5. The bassinet of paragraph A4, wherein the linear-motion actuator is driven by a first pulley and the pair of cams is driven by a second pulley, and a ratio of a cycle of the first pulley to a cycle of the second pulley is approximately 2:1.
A6. The bassinet of any one of paragraphs A0 through A5, wherein the transducer comprises a bass shaker.
A7. The bassinet of paragraph A6, wherein the transducer is configured to produce vibrations within substantially an entirety of the movable platform.
B0. A system for transitioning an infant from an intrauterine environment, the system comprising a low-frequency transducer coupled to an underside of a platform and configured to transmit low-frequency sound waves and/or vibrations through the platform to an upper side of the platform opposite the underside; and a motion-control system configured to translate the platform along a longitudinal axis of the platform and to rotate the platform about the longitudinal axis.
B1. The system of paragraph B0, further comprising a plurality of vibration-isolating components configured to vibrationally isolate the platform from the motion-control system.
B2. The system of paragraph B1, wherein the vibration-isolating components comprise low-durometer elastomeric isolation grommets.
B3. The system of any one of paragraphs B0 through B2, wherein the low-frequency transducer is configured to produce vibrations having frequencies below approximately 80 Hz.
B4. The system of paragraph B3, wherein the low-frequency transducer is configured to transmit low-frequency waves having a fundamental frequency no greater than approximately 50 Hz.
B5. The system of any one of paragraphs B3 through B4, wherein the waves transmitted by the low-frequency transducer have an aggregate energy, and a majority of the aggregate energy of the waves is associated with components of the waves having frequencies below approximately 80 Hz.
B6. The system of any one of paragraphs B0 through B5, further comprising a mid-range transducer coupled to the underside of the platform and configured to produce sound waves and/or vibrations having frequencies predominantly above approximately 100 Hz.
C0. An infant support system comprising a platform defining a longitudinal axis; means for translating the platform along the longitudinal axis; means for rotating the platform about the longitudinal axis; and means for inducing low-frequency acoustic waves and/or vibrations in the platform.
C1. The system of paragraph C0, wherein the means for translating the platform include slide shaft driven by a motor.
C2. The system of paragraph C1, wherein the means for rotating the platform include a pair of offset cams mounted to a shaft driven by the motor.
C3. The system of any one of paragraphs C0 through C2, wherein the means for inducing low-frequency acoustic waves and/or vibrations in the platform include a bass shaker configured to produce acoustic waves predominantly in a range of approximately 20 to 80 Hz.
C4. The system of paragraph C3, further comprising means for vibrationally isolating the tactile transducer from the means for translating the platform and from the means for rotating the platform.

### Advantages, Features, and Benefits

The different embodiments and examples of the infant support system described herein provide several advantages over known solutions for simulating an intrauterine environment. For example, illustrative embodiments and examples described herein allow a simulation of a maternal heartbeat that includes generation of acoustic waves and/or vibrations at frequencies characteristic of the maternal heartbeat as experienced by a fetus inside the uterus.

Additionally, and among other benefits, illustrative embodiments and examples described herein allow an infant to experience a simulated maternal heartbeat as a substantially full-body vibration associated with conduction through bone rather than as sound waves associated with the ears.

Additionally, and among other benefits, illustrative embodiments and examples described herein allow simulation of heartbeat sensations and motion associated with the intrauterine environment to be tapered off over time, thereby facilitating an infant's transition from the intrauterine environment to the extrauterine environment.

Additionally, and among other benefits, illustrative embodiments and examples described herein allow a modular intrauterine simulator unit to be easily installed and removed from a bassinet or similar device, allowing the bassinet to be used in a conventional manner before the simulator unit is installed and after the simulator unit is removed. This allows the simulator unit to be reused in another bassinet (e.g., for another infant). The ability to reuse the simulator unit may be convenient, e.g., because a given infant typically uses the simulator unit for a relatively short time (e.g., months). A removable, reusable unit may be borrowed and/or rented for the duration of time during which it is beneficial to the infant, and then passed on to another infant who is ready to benefit from it.

Additionally, and among other benefits, illustrative embodiments and examples described herein include a robust suspension system capable of withstanding a long period of use (e.g., capable of sequentially and/or serially being used with a plurality of infants), and able to be shipped a plurality of times (e.g., between a plurality of homes, hospitals, and/or distribution centers) without sustaining damage or requiring excessive care.

Additionally, and among other benefits, illustrative embodiments and examples described herein allow a modular, self-contained device having motion-control and/or heartbeat-simulation systems. This removable core module may be configured to be easily installed in and removed from a plurality of different settings (e.g., bassinets, cushions, carriers, beds, and/or the like). For example, the removable core module is removable without tools, and may be removable with a single hand. Additionally, in some examples the removable core module is configured to be installed at a convenient and/or adjustable height in a bassinet. This may make use of the device convenient and flexible.

Additionally, and among other benefits, illustrative embodiments and examples described herein allow simulation of motion patterns characteristic of those experienced by a fetus during the third trimester of gestation. In contrast, known systems typically are configured only for a general rocking pattern. Accordingly, illustrative embodiments and examples described herein simulate the intrauterine experience much more closely than known systems.

Additionally, and among other benefits, illustrative embodiments and examples described herein allow production of motion quietly and smoothly, with high safety and reliability and very low maintenance.

No known system or device can perform these functions. However, not all embodiments and examples described herein provide the same advantages or the same degree of advantage.

### Conclusion

The disclosure set forth above may encompass multiple distinct examples with independent utility. Although each of these has been disclosed in its preferred form(s), the specific embodiments thereof as disclosed and illustrated herein are not to be considered in a limiting sense, because numerous variations are possible. To the extent that section headings are used within this disclosure, such headings are for organizational purposes only. The subject matter of the disclosure includes all novel and nonobvious combinations and subcombinations of the various elements, features, functions, and/or properties disclosed herein. The following claims particularly point out certain combinations and subcombinations regarded as novel and nonobvious. Other combinations and subcombinations of features, functions, elements, and/or properties may be claimed in applications claiming priority from this or a related application. Such claims, whether broader, narrower, equal, or different in scope to the original claims, also are regarded as included within the subject matter of the present disclosure.

1. A bassinet configured to simulate an intrauterine environment, the bassinet comprising:
a movable platform configured to support a floor of the bassinet, the movable platform defining a longitudinal axis;
a suspension system including:
at least one linear-motion actuator attached to a fixed frame disposed underneath the movable platform and configured to linearly displace the movable platform along the longitudinal axis; and
at least one rotational-motion actuator configured to impart rotational motion to the movable platform;
a drive system configured to drive the suspension system; and
a transducer disposed underneath the movable platform and configured to produce low frequency vibrations;
wherein the at least one rotational-motion actuator of the suspension system comprises a pair of cams attached to a shaft disposed transverse to the longitudinal axis, and wherein the cams are offset from each other.

2. The bassinet of claim 1, wherein the movable platform, the suspension system, and the drive system are configured to be removable from the bassinet.

3. The bassinet of claim 1, wherein the drive system includes an electronic controller configured to vary the linear displacement and rotational motion based on a time of day.

4. (canceled)

5. The bassinet of claim 1, wherein the drive system is configured to drive the linear-motion actuator and the pair of cams to move the movable platform in a modified figure-eight pattern.

6. The bassinet of claim 5, wherein the linear-motion actuator is driven by a first pulley and the pair of cams is driven by a second pulley, and a ratio of a cycle of the first pulley to a cycle of the second pulley is approximately 2:1.

7. The bassinet of claim 1, wherein the transducer comprises a bass shaker.

8. The bassinet of claim 7, wherein the transducer is configured to produce vibrations in the range of approximately 20 to 80 Hz.

9. A system for transitioning an infant from an intrauterine environment, the system comprising:
a low-frequency transducer coupled to an underside of a platform and configured to transmit low-frequency sound waves and vibrations through the platform to an upper side of the platform opposite the underside;
a motion-control system configured to translate the platform along a longitudinal axis of the platform and to rotate the platform about the longitudinal axis; and
a plurality of vibration-isolating components configured to vibrationally isolate the platform from the motion-control system, wherein the vibration-isolating components comprise low-durometer elastomeric isolation grommets.

10-11. (canceled)

12. The system of claim 9, wherein the low-frequency transducer is configured to produce low frequency vibrations.

13. The system of claim 12, wherein the low-frequency transducer is configured to produce vibrations in the range of approximately 20 to 80 Hz.

14. The system of claim 12, wherein the sound waves and vibrations transmitted by the low-frequency transducer have an aggregate energy, and a majority of the aggregate energy of the sound waves and vibrations is associated with components of the sound waves having frequencies below approximately 80 Hz.

15. The system of claim 9, further comprising a mid-range transducer coupled to the underside of the platform and configured to produce sound waves and vibrations having frequencies predominantly above approximately 100 Hz.

16. An infant support system comprising:
a platform defining a longitudinal axis;
means for translating the platform along the longitudinal axis;
means for rotating the platform about the longitudinal axis; and
means for inducing low-frequency acoustic waves and vibrations in the platform;
wherein the means for translating the platform include a slide shaft driven by a motor; and
wherein the means for rotating the platform include a pair of offset cams mounted to a shaft driven by the motor.

17-18. (canceled)

19. The system of claim 16, wherein the means for inducing low-frequency acoustic waves and vibrations in the platform include a bass shaker configured to produce acoustic waves and vibrations predominantly in a range of approximately 20 to 80 Hz.

20. The system of claim 19, further comprising means for vibrationally isolating the tactile transducer from the means for translating the platform and from the means for rotating the platform.
